(19) United States
(12) Patent Application Publication
(10) Pub. No.: US 2026/0140059 A1
(43) Pub. Date: May 21, 2026

(54) METHOD AND MOBILE DEVICE FOR DETECTING AN ANALYTE IN A SAMPLE OF A BODILY FLUID

(71) Applicant: Roche Diabetes Care GmbH, Mannheim (DE)

(72) Inventors: Bernhard Limburg, Soergenloch (DE); Max Berg, Mannheim (DE); Fredrik Hailer, Limburgerhof (DE); Susana Contreras, Berlin (DE); Volker Tuerck, Berlin (DE)

(21) Appl. No.: 19/445,180

(22) Filed: Jan. 9, 2026

Related U.S. Application Data
(63) Continuation of application No. PCT/EP2024/068681, filed on Jul. 3, 2024.

(30) Foreign Application Priority Data
Jul. 12, 2023 (EP) 23 185 069.4

(51) Int. Cl. G01N 21/78; G06T 7/00

(52) U.S. Cl. CPC G01N 21/78; G06T 7/0014; G01N 2201/0221; G06T 2207/10004; G06T 2207/30004

(57) ABSTRACT

A method of detecting an analyte in a body fluid sample using a mobile device having a camera. An image is obtained with the camera of a test field having the body fluid sample applied thereto, causing an optical detection reaction. An algorithm is then performed to determine a potential detection of an optical detection feature and a potential item of intensity information. The potential item of intensity information is weighted with a known item of position probability information. The known item of position probability information is a probability of a potential item of intensity information on the optical detection reaction being a valid item of intensity information. The analyte is detected considering the weighted potential item of intensity information for differentiating a valid from an invalid item of intensity information. A calibration process is then performed in which the known item of position probability information is selected.

### RELATED APPLICATIONS

This application is a continuation of International Patent Application No. PCT/EP2024/068681, filed Jul. 3, 2024, which claims priority to EP 23 185 069.4, filed Jul. 12, 2023, both of which are hereby incorporated herein by reference.

### BACKGROUND

This disclosure relates to a computer-implemented method of detecting at least one analyte in a sample of a bodily fluid by using a mobile device having at least one camera and a mobile device having at least one camera. This disclosure further relates to a corresponding computer program and a computer-readable storage medium, as well as to a corresponding analytical system. This disclosure specifically may be used in medical diagnostics in order to quantitatively or qualitatively detect one or more properties of a sample of a bodily fluid. As an example, the methods and devices may be used for qualitatively and/or quantitatively detecting the presence of one or more analytes in a sample of a bodily fluid, such as for detecting glucose. Additionally or alternatively, as an example, this disclosure may be used for detecting the presence of the SARS-CoV-2 coronavirus in a sample of a bodily fluid. Other fields of application of this disclosure, however, are also feasible.

In the field of medical diagnostics, in many cases, one or more analytes have to be detected in samples of a body fluid, such as blood, interstitial fluid, urine, saliva or other types of body fluids. Examples of analytes to be detected are viruses, such as the SARS-CoV-2 coronavirus. However, this disclosure may also be used for other types of analytes, such as glucose, triglycerides, lactate, cholesterol or other types of analytes, that, typically, are present in these body fluids. According to the concentration and/or the presence of the analyte, an appropriate treatment may be chosen, if necessary.

Generally, devices and methods known to the skilled person make use of test elements comprising one or more test chemicals, which, in presence of the analyte to be detected, are capable of performing one or more detectable detection reactions, such as optically detect-able detection reactions. As an example, in the field of glucose detection, with regard to the test chemicals comprised in test elements, reference may be made e.g., to J. Hoenes et al.: The Technology Behind Glucose Meters: Test Strips, Diabetes Technology & Therapeutics, Volume 10, Supplement 1, 2008, S-10 to S-26. Other types of test chemistry are possible and may be used for performing this disclosure.

As a further example that may be of particular interest for this disclosure, for point-of-care (POC) testing or for other situations where a quick result is desirable, a variety of lateral flow (LF) tests detecting viral antigens are commercially available. For example, in the case of SARS-CoV-2, LF test detecting the nucleocapsid antigen are widely used, but also the spike antigen may be detected. However, also tests detecting viral nucleic acids have been proposed as lateral flow tests, cf., e.g., WO 2021/228839 and references cited therein, which are also referred to as “SHERLOCK” tests. Further, instead of blood tests, saliva was proposed as a suitable sample material for SARS-CoV-2 detection (Wyllie et al. (2020), medRxiv 2020.04.16.20067835; doi.org/10.1101/2020.04.16.20067835). B. Ince and M. K. Sezgintürk provide in “Lateral flow assays for viruses diagnosis: Up-to-date technology and future prospects,” 2022, TrAC Trends in Analytical Chemistry, Volume 157, 116725, an overview of current problems and accessible solutions in detecting infectious agents and diseases by lateral flow assay (LFA), focusing on increasing sensitivity with various detection methods. Further, the sensitivity of SARS-CoV-2 antigen rapid diagnostic tests (Ag RDT) was evaluated in “Comparative sensitivity evaluation for 122 CE-marked rapid diagnostic tests for SARS-CoV-2 antigen, Germany, September 2020 to April 2021,” H. Scheiblauer et al., Eurosur-veillance, 26, 2100441 (2021).

Typically, the detection of the analyte may rely on an optical detection reaction that becomes visible on an optical test element to indicate the presence of the analyte. When using a mobile device for detecting the analyte, particularly in home-testing scenarios, images of the optical test element may be captured by a camera comprised by the mobile device. The detection of the analyte may then be based on a computer-implemented analysis of the images. The detection of the visible optical detection reaction may be performed considering the full image or at least one defined region of interest on the image.

A technical challenge may lie in the circumstance that these images may contain artifacts, such as unwanted distortions or anomalies that may occur during the image acquisition and/or processing, which are difficult to distinguish from the visible optical detection reaction. In case the optical detection reaction represents a line feature, a line detection algorithm may exemplarily be used and a line on the image may be considered as visible optical detection reaction. There may be cases, in which the line detection algorithm cannot clearly determine whether there is a line that is associated with an optical detection reaction or an artifact. This may lead to false positive detections of the analyte, particularly in case an artifact may be classified and/or identified as a visible optical detection reaction. Also features in the image, such as shadows or the like, may lead to false positive detections by image recognition algorithms.

U.S. Pat. No. 11,275,020 B2 discloses a lateral flow assay device including several markers, a color bar, and/or a grayscale on the housing of the lateral flow assay device. The markers are used to assist in focusing a mobile device's camera on the control line of the lateral flow assay device. The markers may be used to adjust the perspective of an image taken from the control line and the test line of the lateral flow assay device. The markers may be used to locate the images of the control line, the test line, the color bar, and/or the grayscale on the image. The image of the color bar and the grayscale may be used to adjust the colors and intensity of the image. The images of the test line and the control line may then be used to determine the test results of the lateral flow assay device.

U.S. Pat. No. 11,494,571 B2 discloses embodiments that are generally directed to improving feature detection of rapidly acquired images using camera-enabled mobile devices involving a 2-D decal code, such as a QR code, for improving the reading accuracy of a rapid diagnostic antigen or antibody or enzymatic colorimetric directed test, such as for COVID-19 diagnosis. One primary issue with evaluating a Covid-19 rapid test is detecting and quantifying positive test lines from sampled test strips based on digital images of the test strip. Aspects of this disclosure contemplate masking a QR code to improve the sample image resolution and contrast. Other aspects of this disclosure contemplate methods and techniques to evaluate a test line on the sample image by enhancing an intensity curve along the test line and control line containing area by way of calculating the instantaneous change in pixel intensity and evaluating the position and intensity of those signals.

U.S. Publication No. 2013/0273563 A1 discloses enzyme-based diagnostic testing systems for detecting and quantifying at least one of the activity level or the concentration of an enzyme or a biochemical analyte in a biological sample. Such enzyme-based diagnostic testing systems can provide rapid, accurate, affordable laboratory-quality testing at the point of care. An enzyme-based diagnostic testing system may include a lateral-flow chromatographic assay cassette that is configured for assaying an amount or activity of an enzyme in a sample or for enzymatically determining the concentration of an enzyme substrate in a sample. Additionally, the enzyme-based diagnostic testing systems may include testing devices (e.g., a smartphone or a similar remote computing device) having data collection and data analysis capabilities. Such testing devices may also include automated data reporting and decision support.

WO 2020/016616 A1 discloses a portable, rechargeable lateral flow test strip immunoassay in vitro diagnostic device including a color imaging sensor for imaging a lateral flow test strip, a lens that images a substantial part of the 2D surface of the lateral flow test strip onto the color imaging sensor and image processing software that identifies patterns in the test lines and/or dots or other distributions in the test strip and compensates for distortions, non-uniformities or anomalies in the lines and/or dots or other distributions in the test strip.

EP 3 791 167A 1 discloses an analyte testing system for quantifying the presence of an analyte in a specimen by lateral flow chromatography. The system comprises a test cassette with a lateral flow chromatography and a mobile hand-held processor device comprising a digital camera, a source of light and a processor, which software and hardware are configured to determine automatically the distance between camera and object and the measures of light in the region of interest of the lateral flow chromatography prior any retrieval of image data for further analysis and quantification of the visual signals.

U.S. Publication No. 2022/0254133 A1 discloses a computer implemented method for reading a test region of an assay, the method comprising: (i) providing digital image data of a first assay; (ii) inputting the digital image data into a trained convolutional neural network configured to output a first probability, based on the input digital image data, that a first region of pixels of the digital image data corresponds to a first test region of the assay; (iii) if the first probability is at or above a first predetermined threshold, accepting the first region of pixels as a first region of interest associated with the first test region; and (iv) estimating an intensity value of a portion of the first test region in the first region of interest.

WO 2022/049441 A1 discloses a mobile computing device that includes camera hardware configured to capture image data associated with an output signal area of a biological chromatographic test strip. The mobile computing device further includes processing circuitry in communication with the camera hardware, the processing circuitry being configured to determine, based on the image data captured by the camera hardware, a concentration of a target analyte in a test sample submitted via the biological chromatographic test strip. The mobile computing device further includes an inter-face in communication with the processing circuitry, the interface being configured to output data indicative of the concentration of the target analyte determined by the processing circuitry.

U.S. Pat. No. 8,916,390 B2 discloses a portable rapid diagnostic test reader system including a mobile phone having a camera and one or more processors contained within the mobile phone and a modular housing configured to mount to the mobile phone. The modular housing including a receptacle configured to receive a sample tray holding a rapid diagnostic test. At least one illumination source is disposed in the modular housing and located on one side of the rapid diagnostic test. An optical demagnifier is disposed in the modular housing interposed between the rapid diagnostic test and the mobile phone camera.

To prevent false positive detections of the analyte, particularly caused by artifacts or falsely identified features in the image, typically, the sensitivity on the detection of the analyte, such as by increasing the detection threshold, is sacrificed.

### SUMMARY

This disclosure teaches a computer-implemented method of detecting at least one analyte in a sample of a bodily fluid by using a mobile device having at least one camera and a mobile device having at least one camera, which at least partially address the above-mentioned challenges. Specifically, this disclosure teaches a computer-implemented method of detecting at least one analyte in a sample of a bodily fluid by using a mobile device having at least one camera showing an increased detection threshold, particularly with a low rate of false positive detections, for the at least one analyte.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “analyte,” “camera,” and “image,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a computer-implemented method of detecting at least one analyte in a sample of a bodily fluid by using a mobile device having at least one camera is disclosed. For details, options and definitions, reference may be made to any aspect, embodiment or claim discussed elsewhere herein.

The computer-implemented method of detecting the at least one analyte comprises the following steps, which may be performed in the given order. A different order, however, may also be feasible. Further, two or more of the method steps may be performed simultaneously. Thereby the method steps may at least partly overlap in time. Further, the method steps may be performed once or repeatedly. Thus, one or more or even all of the method steps may be performed once or repeatedly. The method may comprise additional method steps, which are not listed herein.

The term “computer implemented” as used herein is a broad term and is to be given its ordinary and customary meaning to a person of ordinary skill in the art and is not to be limited to a special or customized meaning. The term specifically may refer, without limitation, to a method which is performed by using computer programming, and/or by using at least one computer and/or at least one computer network. Thus, as an example, one or more or even all of the method steps may be performed by appropriate software, e.g., by using computer-readable instructions which, when executed on a computer or a computer network, cause the computer or computer network to perform the method steps. The term “software” as used herein may, specifically, refer to a computer program. The computer and/or computer network may comprise at least one processor, which is configured for performing at least one, more than one or all of the method steps of the method according to this disclosure. Specifically, each of the method steps is performed by the computer and/or computer network. The method may be performed completely automatically, specifically without user interaction.

The term “mobile device” as used herein is a broad term and is to be given its ordinary and customary meaning to a person of ordinary skill in the art and is not to be limited to a special or customized meaning. The term specifically may refer, without limitation, to a mobile electronics device, specifically a personal mobile device (PDA), more specifically to a mobile communication device such as a cell phone and/or a smartphone. Additionally or alternatively, the mobile device may also refer to a notebook, a tablet computer or another type of portable computer, such as a wearable, specifically smart glasses, having at least one camera. Alternatively or in addition, a smartphone having an external camera may be used. The external camera may be comprised by spectacles. The mobile device may have a direct internet access, particularly in a manner that the mobile device is not required of being required to connect to a network, such as a wireless local area network (LAN) network, for connecting to the internet. Thus, generally, the mobile devices may be selected from the group consisting of: a cell phone having at least one camera, specifically a smart phone; a portable computer having at least one camera, specifically at least one of a notebook and a tablet computer. The mobile device may comprise a processor, particularly for performing the computer implemented method.

The method comprises the following steps:
a. obtaining image data of at least one image captured by using the camera, wherein the image comprises at least a part of a test field of an optical test element having the sample of the bodily fluid applied thereto, wherein the test field is configured for performing at least one optical detection reaction in the presence of the analyte in the sample of the bodily fluid, wherein the optical detection reaction is visible at at least one predetermined position on the test field;
b. performing at least one image recognition algorithm on the image, wherein the image recognition algorithm is configured for determining a potential detection of at least one optical detection feature of at least one optical detection reaction in the image, wherein the image recognition algorithm is configured for determining at least one potential item of intensity information on the optical detection feature of the optical detection reaction in the image and at least one associated item of position information on the optical detection feature of the optical detection reaction in the image;
c. weighting the potential item of intensity information on the optical detection feature of the optical detection reaction with at least one known item of position probability information on the optical detection reaction, wherein the known item of position probability information is a probability of a potential item of intensity information on the optical detection reaction to be a valid item of intensity information on the optical detection reaction depending on the associated item of position information on the optical detection, wherein the known item of position probability information on the optical detection reaction is determined by considering the associated item of position information on the optical detection reaction, wherein the at least one known item of position probability information considers at least one tolerance in a manufacturing process of the optical test element having an influence on the at least one predetermined position of the optical detection reaction on the test field;
d. detecting the analyte by considering the weighted potential item of intensity information on the optical detection reaction for differentiating a valid item of intensity information on the optical detection reaction from an invalid item of intensity information, particularly associated with false positive object.

In a step a., image data of at least one image captured by using the camera is obtained, wherein the image comprises at least a part of a test field of an optical test element having the sample of the bodily fluid applied thereto, wherein the test field is configured for performing at least one optical detection reaction in the presence of the analyte in the sample of the bodily fluid, wherein the optical detection reaction is visible at at least one predetermined position on the test field.

The term “image data” as used herein is a broad term and is to be given its ordinary and customary meaning to a person of ordinary skill in the art and is not to be limited to a special or customized meaning. The term specifically may refer, without limitation, to a collection of information representing visual content in a digital format. Typically, image data may comprise one or more pixels. The one or more pixels may be arranged in a known manner, particularly a known grid. Any one of the one or more pixels may comprise at least one numerical value defining a color and/or an intensity of the respective pixel. The numerical value may be binary data and/or data of a known color model, such as RGB (Red, Green, Blue) or CMYK (Cyan, Magenta, Yellow, Black). Alternatively or in addition, the image data may be vector image data. The vector image data may be at least one of: one or more points, one or more lines, one or more curves, and one or more further geometric elements.

The term “obtaining” as used herein is a broad term and is to be given its ordinary and customary meaning to a person of ordinary skill in the art and is not to be limited to a special or customized meaning. The term specifically may refer, without limitation, to the procedure of getting and/or becoming access to data. Obtaining may comprise receiving the data from a sending device, such as a mobile device. For receiving the data, obtaining may comprise requesting the data from the mobile device, such as by sending a query to the mobile device. The data may be generated by using the mobile device, such as by capturing the image by using the camera of the mobile device, specifically the mobile device. Particularly in this case, the steps a., b., c. and/or d. may be performed by using a server and/or a cloud server. Alternatively or in addition, obtaining may comprise generating the data, such as by capturing the image by using the camera of the mobile device. The method may then comprise a step of capturing the at least one image by using the camera. Particularly in this case, the steps a., b., c. and/or d. may be performed by using the mobile device.

The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, two-dimensional or even three-dimensional optical data or information. As an example, the camera may comprise at least one camera chip, such as at least one CCD chip and/or at least one CMOS chip configured for recording images. As used herein, without limitation, the term “image” specifically may relate to data recorded by using the camera, such as a plurality of electronic readings from the imaging device, such as the pixels of the camera chip.

The camera, besides the at least one camera chip or imaging chip, may comprise further elements, such as one or more optical elements, e.g., one or more lenses. As an example, the camera may be a fix-focus camera, having at least one lens which is fixedly adjusted with respect to the camera. Alternatively, however, the camera may also comprise one or more variable lenses which may be adjusted, automatically or manually. This disclosure specifically shall be applicable to cameras as usually used in mobile applications, such as notebook computers, tablets or, specifically, cell phones such as smartphones. Thus, specifically, the camera may be part of the mobile device which, besides the at least one camera, comprises one or more data processing devices such as one or more data processors. Other cameras, however, are feasible.

The camera specifically may be a color camera. Thus, such as for each pixel, color information may be provided or generated, such as color values for three colors R, G, B. A larger number of color values is also feasible, such as four colors for each pixel, for example, R, G, G, B. Color cameras are generally known to the skilled person. Thus, as an example, each pixel of the camera chip may have three or more different color sensors, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used, such as R, G, G, B or C, M, Y, K or the like. The color sensitivities of the pixels may be generated by color filters or by appropriate intrinsic sensitivities of the sensor elements used in the camera pixels. These techniques are generally known to the skilled person.

The term “capturing at least one image” as used herein is a broad term and is to be given its ordinary and customary meaning to a person of ordinary skill in the art and is not to be limited to a special or customized meaning. The term specifically may refer, without limitation, to one or more of imaging, image recording, image acquisition, image capturing. The capturing at least one image may comprise recording a single image and/or a plurality of images such as a sequence of images. For example, the recording of the image may comprise recording continuously a sequence of images such as a video or a movie. The recording of the at least one image may be initiated by a user action or may automatically be initiated, e.g., once the presence of the at least one object within a field of view and/or within a predetermined sector of the field of view of the camera is automatically detected. These automatic image acquisition techniques are known, e.g., in the field of automatic bar-code readers, such as from automatic barcode reading apps. The recording of the images may take place, as an example, by acquiring a stream or “live stream” of images with the camera, wherein one or more of the images, automatically or by user interaction such as pushing a button, are stored and used as the at least one first image or the at least one second image, respectively. The image acquisition may be supported by the processor of the mobile device.

The term “sample of a bodily fluid” as used herein is a broad term and is to be given its ordinary and customary meaning to a person of ordinary skill in the art and is not to be limited to a special or customized meaning. The term specifically may refer, without limitation, to an arbitrary aliquot part or aliquant part of a biological fluid which directly is a bodily fluid or which is derived from a bodily fluid, such as by one or more pre-processing steps, e.g., by transferring a bodily fluid to at least one sampling fluid, by diluting a bodily fluid, by centrifugation a bodily fluid or the like. The bodily fluid may comprise one or more of saliva, blood, interstitial fluid, urine or other types of body fluids. The sample of the bodily fluid may be collected via at least one nasopharyngeal swab, at least one swab of the anterior nares or from saliva, such by applying a cotton swab to a surface of the anterior nares and/or the throat. The collected sample of bodily fluid may be transferred to at least sampling or reagent fluid by immersing the cotton swab in the sampling or reagent fluid. The sampling or reagent fluid may specifically comprise lysis reagents. Alternatively or additionally, the sample of the bodily fluid may be a droplet of a body fluid as gathered from the body of a person, such as a droplet of saliva and/or blood and/or interstitial fluid or the like. The sample of the bodily fluid may specifically comprise at least one preparation of the bodily fluid, such as a cell preparation of the bodily fluid, e.g., a stained cell preparation of the bodily fluid. The sample of bodily fluid may also be simply referred to as the sample or the test sample.

As used herein, the term “optical detection” refers to a detection of a reaction using an optical test chemical, such as a color-change test chemical, which changes in color in the presence of the analyte. The color change specifically may depend on the amount of analyte present in the sample. Techniques for detecting the analyte by optical detection and in particular analyzing color of the spot on the test filed are generally known to the skilled person. The optical detection reaction may comprise a color formation reaction. The term “color formation reaction” as used herein is a broad term and is to be given its ordinary and customary meaning to a person of ordinary skill in the art and is not to be limited to a special or customized meaning. The term specifically may refer, without limitation, to a chemical, biological or physical reaction during which a color, specifically a reflectance, of at least one element involved in the reaction, changes with the progress of the reaction.

The term “optical test element” as used herein is a broad term and is to be given its ordinary and customary meaning to a person of ordinary skill in the art and is not to be limited to a special or customized meaning. The term specifically may refer, without limitation, to an arbitrary element or device configured for performing an optical detection reaction, for example, a color-change detection reaction and/or a reaction during which one or more optically detectable features on or within the test element become visible, such as one or more markings, such as linear markings known from rapid COVID testing. The optical test element may, as an example, be embodied as a test stick or as a test element.

The optical test element may be a digital-type test element for digitally detecting the presence or absence of at least one predetermined analyte in the sample. Thus, the optical test element may be a test element capable of providing the information “positive” if the at least one predetermined analyte is determined to be present in the sample, or “negative” if the at least one predetermined analyte is determined not to be present in the sample. For determining the information “positive” and/or “negative” at least one of: the item of intensity information; the weighted item of intensity information may be considered. Preferably for determining the information “positive” and/or “negative,” the weighted item of intensity information may be considered. The optical test element may be configured for changing at least one optically detectable property of at least one feature when the analyte is detected in the sample, whereas the at least one optically detectable property of the at least one feature may be kept unchanged when the analyte is not detected in the sample. As an example, the optical test element may be a SARS-CoV-2 rapid antigen test, specifically a SARS-CoV-2 & Flu A/B rapid antigen test.

The optical detection reaction may comprise a reaction during which one or more optical detection features on or within the optical test element becomes visible. The one or more optical detection features may be associated with at least one of:
at least one control area, particularly at a first predetermined position, configured for indicating a correct application of the sample of the bodily fluid on the optical test element;
at least one reagent test region, particularly at a second predetermined position, for detecting the analyte,
   particularly wherein the control area is at a different predetermined position than the reagent test region. The one or more optical detection features may be one or more linear markings.

The term “associated with” as used herein is a broad term and is to be given its ordinary and customary meaning to a person of ordinary skill in the art and is not to be limited to a special or customized meaning. The term specifically may refer, without limitation, to a property of a first element indicating a relationship of the first element with at least one second element with which the first element is associated. The relationship between the first and second elements, as an example, may be at least one of: a relationship by predefinition, a relationship by at least one property the first and second elements have in common, a relationship due to identical or similar causes or origins of the first and second elements, a relationship due to the first element causing or evoking the second element, a relationship due to the second element causing or evoking the first element. A first element may be associated with a second element in a manner that said first element is assigned to said second element, particularly by the image recognition algorithm. The first and second elements, as an example, may be or may comprise optically detectable properties or features which are caused or evoked by the same process, such as by the optical detection reaction, as will be explained in further detail below.

The one or more optical detection features may be associated with at least one control area, in case the one or more optical detection features are related to and/or are the result of an optical detection reaction that indicates the correct application of the sample of the bodily fluid on the optical test element. Said one or more optical detection features may be visible in the control area. The one or more optical detection features may be associated with at least one reagent test region, in case the one or more optical detection features are related to and/or are the result of an optical detection reaction that indicates the detection of the analyte. Said optical detection features may be visible in the reagent test region.

The optical test element may particularly comprise the at least one “control area.” Alternatively or in addition, the optical test element may particularly comprise the at least one “reagent test region” containing at least one test chemical being sensitive for at least one property of the sample, such as for detecting the at least one analyte. The optical test element may, as an example, comprise one or more application sites for applying the at least one sample. The application site may be different from the position of the at least one reagent test region and may be fluidically connected to the reagent test region, such as by one or more capillary elements, such as one or more porous elements capable of transporting liquid. The optical test element, as an example, may comprise at least one substrate, such as at least one carrier, with the at least one reagent test region and/or the control area and/or the application site applied thereto or integrated therein. The optical test element may further comprise a housing. At least a portion of the substrate or the carrier may be arranged inside the housing. The optical test element, particularly the housing, may further comprise a window through which access to the substrate may be possible, particularly for applying the sample and/or visually inspecting the optical detection reaction.

The “control area” may be arranged in a proximity to the reagent test region. The control area may be, for example, enclosing or surrounding the reagent test region and/or may be arranged behind the reagent test region in a direction of flow of the sample of bodily fluid on the optical test element. The control area may be a separate field independently arranged on the substrate or carrier. The carrier, as an example, may be strip-shaped, thereby rendering the optical test element a test strip. These test strips are generally widely in use and available. One test strip may carry a single reagent rest region or a plurality of reagent test regions having identical or different test chemicals comprised therein. Additionally or alternatively, the optical test element may be embodied as a stick or chip, e.g., with a housing having the above-mentioned substrate disposed therein, e.g., a housing having one or more application openings for applying the sample and one or more detection windows for enabling an optical detection of the at least one detection reaction. Various options are feasible.

The term “reagent test region” as used herein is a broad term and is to be given its ordinary and customary meaning to a person of ordinary skill in the art and is not to be limited to a special or customized meaning. The term specifically may refer, without limitation, to a coherent amount of the test chemical, such as to a field, e.g., a field of round, polygonal, line or rectangular shape, having one or more layers of material, with at least one layer of the reagent test region having the test chemical comprised therein. Other layers may be present providing specific optical properties such as reflective properties, providing spreading properties for spreading the sample or providing separation properties such as for separating of particulate components of the sample, such as cellular components. The sample of the bodily fluid may be applied directly to the reagent test region, e.g., for blood glucose measurements where a droplet of blood may be directly applied to the test strip comprising the reagent test region, or may be applied indirectly to the reagent test region, e.g., by applying the sample of the bodily fluid to a reservoir or an application site of the optical test element, wherein the sample of the bodily fluid may flow from the reservoir of application site to the reagent test region of the optical test element, such as by capillary forces acting on the sample of the bodily fluid.

As already discussed, the optical detection reaction is visible at at least one “predetermined position on the test field.” Typically, the predetermined position may be a nominal position, particularly a known nominal position that may, exemplarily, be provided by the manufacturer of the optical test element. The predetermined position may be influenced by the manufacturing process of the optical test element. Typically, the predetermined position may depend on the tolerances of the manufacturing process.

In a step b., at least one image recognition algorithm on the image is performed, wherein the image recognition algorithm is configured for determining at least one potential item of intensity information on the optical detection reaction in the image and at least one associated item of position information on the optical detection reaction in the image.

The term “image recognition” as used herein is a broad term and is to be given its ordinary and customary meaning to a person of ordinary skill in the art and is not to be limited to a special or customized meaning. The term specifically may refer, without limitation, to a process of recognizing one or more features in an image. The image recognition may be considering the image data. Specifically, the image recognition may be configured for visually identifying the visual optical detection reaction, specifically the optical detection features, such as the control area and/or the reagent rest region. Thereby, the image recognition algorithm may determine at least one valid feature associated with the visible optical detection reaction and/or at least one invalid feature not associated with the visible optical detection reaction. The invalid feature may be associated with a false positive.

The term “intensity” as used herein is a broad term and is to be given its ordinary and customary meaning to a person of ordinary skill in the art and is not to be limited to a special or customized meaning. The term specifically may refer, without limitation, to at least one of: an amount, a strength, a magnitude of at least one characteristic, specifically of a feature. The intensity may refer to a degree of visible change associated with the occurrence of the optical detection reaction. The intensity, particularly the degree of visible change, may be an indication of the concentration of the analyte in the sample. The intensity, particularly the degree of visible change, may be correlated, specifically positively correlated, to the concentration of the analyte in the sample. Exemplarily, a large degree of visible change may indicate a high concentration the visible change and a small degree of visible change may indicate a low concentration the visible change. Detecting the analyte may comprise determining at least one concentration of the analyte. For determining the at least one concentration of the analyte the item of intensity information may be considered.

Method steps for determining a concentration of the analyte may comprise the following steps, which may be performed in the given order. A different order, however, may also be feasible. Further, two or more of the method steps may be performed simultaneously. Thereby the method steps may at least partly overlap in time. Further, the method steps may be performed once or repeatedly. Thus, one or more or even all of the method steps may be performed once or repeatedly.

At least one, preferably any one, of the method steps may be computer-implemented. At least one, preferably any one, of the method steps may be performed by using a processing unit. Alternatively or in addition, at least one, preferably any one, of method steps may be performed by the mobile device and/or the server, specifically the cloud server.

The method steps may be performed as part of the method computer-implemented method of detecting the at least one analyte in a sample of a bodily fluid by using the mobile device having the at least one camera, specifically the method steps may be performed in method step d. Alternatively, the method steps may be performed as part of an independent method for determining the concentration of the analyte.

The method steps for determining the concentration of the analyte may comprise:
clearing the image data of the at least one obtained image captured by using the camera of at least one false positive, specifically wherein the at least one false positive is or comprises at least one of: at least one artifact in the image; at least one falsely identified feature in the image,
   optionally wherein clearing the image data is performed by at least one of:
      considering, particularly exclusively, the at least one valid item of intensity information on the optical detection reaction when determining the concentration;
      discarding and/or rejecting the at least one invalid item of intensity information;
      discarding and/or rejecting at least one a portion of the image data, particular of image data related to the at least one false positive,
normalizing a brightness of the image data to one or more average brightnesses of a specific area of the image area, specifically a white area of interest, such as a featureless area between the at least one control area and the at least one at least one reagent test region;
determining an intensity value I(test) of the item of intensity information on the optical detection reaction, particularly the optical detection reaction of the optical detection feature of the at least one reagent test region,
   optionally wherein the intensity value I(test) of the item of intensity information is determined by at least one of:
      averaging the item of intensity information, such as by integrating over the item of intensity information;
      selecting the maximum intensity value of the item of intensity information,
optionally determining a further intensity value I(control) of the item of intensity information on the optical detection reaction, particularly the optical detection reaction of an optical detection feature of the at least one control area,
   further optionally wherein the further intensity value I(control) of the item of intensity information is determined by at least one of:
      averaging the item of intensity information, such as by integrating over the item of intensity information;
      selecting the maximum intensity value of the item of intensity information,
optionally determining a quotient I(rel)=I(test)/I(control);
determining the concentration of the analyte by considering a function ƒ(I(test)) and/or ƒ(I(rel)), particularly wherein the respective function is an empirical function, more particularly wherein the concentration is the result of the respective function.

The advantage of considering the quotient I(rel) may be an increased robustness against deviations between different batches of the optical test element, particularly introduced due to manufacturing tolerances.

The term “potential item of intensity information” as used herein is a broad term and is to be given its ordinary and customary meaning to a person of ordinary skill in the art and is not to be limited to a special or customized meaning. The term specifically may refer, without limitation, to a candidate for a valid item of intensity information associated with the actual optical detection reaction, particularly as determined by the image recognition algorithm. The potential item of intensity information may be associated with the visible optical detection reaction, specifically a valid feature. Alternatively, the potential item of intensity information may not be associated with the visible optical detection reaction. The potential item of intensity information may be associated with a non-valid feature.

The image recognition algorithm determines a potential detection of at least one optical detection reaction in the image, specifically at least one optical detection feature. The image recognition algorithm may be configured for determining the potential detection of at least one optical detection feature of at least one optical detection reaction in the image by searching for the at least one optical detection feature in the entire test field shown in the image. For the potential detection of an optical detection reaction in the image, the image recognition algorithm may determine a potential item of intensity information on the optical detection reaction, particularly indicating an intensity of the optical detection reaction. Additionally, the image recognition algorithm may determine an associated item of position information on the optical detection reaction, particularly indicating the position of the optical detection reaction. The at least one potential item of intensity information on the optical detection reaction and the at least one associated item of position information on the optical detection reaction may be associated, in case they are both assigned to the same at least one optical detection reaction, particularly by the image recognition algorithm.

The term “position” as used herein is a broad term and is to be given its ordinary and customary meaning to a person of ordinary skill in the art and is not to be limited to a special or customized meaning. The term specifically may refer, without limitation, to a spatial location of at least one point and/or region, particularly given in a multidimensional space such as a two dimensional and/or three dimensional space. The multidimensional space may be described by using coordinates.

The image recognition algorithm may determine the at least one associated item of position information by considering at least one of: a position of a maximum of an intensity distribution, particularly comprised in the potential item of intensity information, a position of a center of an intensity distribution, particularly comprised in the potential item of intensity information, a position of a centroid of the intensity distribution, particularly comprised in the potential item of intensity information, particularly for finding a position of the associated item of position information in reference to the image. The center may be a geometrical center of the intensity distribution. The centroid may be a center of mass of the intensity distribution.

At least one item of position information of a position indicating feature may be determined in the image, particularly by performing the image recognition algorithm, wherein the associated item of position information on the optical detection reaction may be determined by considering the item of position information on the position indicating feature, particularly for finding a position of the associated item of position information in reference to the optical test element. For considering the item of position information on the position indicating feature when determining the associated item of position information on the optical detection reaction, a relative position, particularly a distance, between the item of position information of a position indicating feature and the associated item of position information on the optical detection reaction may be considered.

The item of position information on the position indicating feature may be determined by considering at least one of:
at least one position of at least one marking on the optical test element;
at least one position of at least one geometric feature of the optical test element, particularly at least one feature of a window of the optical test element showing the test field;
at least one position of at least one optical detection feature generated by the optical detection reaction, specifically a position of a control area or a position of a reagent test region;
   comprised by the image.

In a step c., the potential item of intensity information on the optical detection reaction is weighted with at least one known item of position probability information on the optical detection reaction, wherein the known item of position probability information on the optical detection reaction is determined by considering the associated item of position information on the optical detection reaction, wherein the at least one known item of position probability information considers at least one tolerance in a manufacturing process of the optical test element having an influence on the at least one predetermined position of the optical detection reaction on the test field.

The term “weighting” as used herein is a broad term and is to be given its ordinary and customary meaning to a person of ordinary skill in the art and is not to be limited to a special or customized meaning. The term specifically may refer, without limitation, to giving at least one element in a set more influence than at least one further element in the same set. For weighting the potential item of intensity information on the optical detection reaction with the at least one item known position probability information, the potential item of intensity information on the optical detection reaction may be at least one of: convoluted; multiplied; with the known item of position probability information.

The term “known item of position probability information” as used herein is a broad term and is to be given its ordinary and customary meaning to a person of ordinary skill in the art and is not to be limited to a special or customized meaning. The term specifically refers to a probability of a potential item of intensity information on the optical detection reaction to be a valid item of intensity information on the optical detection reaction depending on the associated item of position information on the optical detection. The at least one known item of position probability information may be comprised by a distribution of known position probabilities. The distribution of known position probabilities may have a maximum probability value at an expected or nominal position of the optical detection feature of the optical detection reaction. The expected or nominal position of the optical detection feature of the optical detection reaction may be determined in a calibration process as elsewhere described herein. The maximum probability value may be determined in a calibration process as elsewhere described herein. One or more further probability values comprised by the distribution of known position probabilities may be lower than the maximum probability value. The one or more further probability values may be determined in a calibration process as elsewhere described herein. The distribution of known position probabilities may comprise at least three, four, five or more different probability values. The distribution of known position probabilities may be a continuous function depending on the item of position information on the optical detection feature of the optical detection reaction.

Consequently, the potential item of intensity information and the item of intensity information are determined for a potential detection of at least one optical detection feature and the potential item of intensity information may then be weighted using the distribution of known position probabilities. The result of the weighting process is then subjected to a threshold for determining whether the potential detection of the optical detection feature is a valid optical detection feature or an invalid optical detection feature.

As described above, the at least one known item of position probability considers and/or is based on a tolerance in a manufacturing process of the optical test element. The term “tolerance” as used herein is a broad term and is to be given its ordinary and customary meaning to a person of ordinary skill in the art and is not to be limited to a special or customized meaning. The term specifically may refer, without limitation, to a variation and/or deviation from a specified dimension, parameter and/or characteristic of a product or a component thereof. In the context of this disclosure, the tolerance in the manufacturing process may particularly result in an uncertainty of the predetermined position, particularly introduced in the manufacturing process. Particularly consequently, the predetermined position at which the optical detection reaction occurs may be different for different optical test elements. As an example, an optical test element may comprise a substrate arranged in a housing of the optical test element. Due to the tolerances in the manufacturing process the substrate may be arranged at slightly different positions in different housings. Particularly in respect to markings on the housing and/or features of the housing, the predetermined position of the optical test element may thus be different for different optical test elements. Further, the relative distance between the control area and the reagent test region may deviate for different test sample changing their relative distance.

The at least one known item of position probability information considering the at least one tolerance in the manufacturing process of the optical test element having an influence on the at least one predetermined position on the test field may be determined in a calibration process, particularly a factory calibration process. Alternatively or in addition, the at least one known item of position probability information considering the at least one tolerance in the manufacturing process of the optical test element having an influence on the at least one predetermined position on the test field may be determined by considering at least one known tolerance of one or more, specifically any, steps in the manufacturing process. Considering the at least one known tolerance might be faster than performing the calibration process.

The term “calibration” as used herein is a broad term and is to be given its ordinary and customary meaning to a person of ordinary skill in the art and is not to be limited to a special or customized meaning. The term specifically may refer, without limitation, to the process of adjusting and/or aligning a measurement system in a manner that it generates accurate and/or reliable measurement results. Calibration may involve determining the predetermined position of the optical detection reaction for a plurality of different optical test elements, particularly associated with a same manufacturing batch. For determining the predetermined position of the optical detection reaction in the calibration, a prepared test sample may be applied to the optical test element in a manner that the optical detection reaction occurs and the optical detection reaction may become visible. By considering a plurality of different optical test elements, the known item of position probability information is determined, particularly the distribution of known position probabilities is determined. The term “factory calibration” may refer to the process of calibration being performed by the manufacturer.

In the calibration process at least one item of batch information on the optical test element assigned to the at least one known item of position probability information may be determined. The term “assigned to” as used herein is a broad term and is to be given its ordinary and customary meaning to a person of ordinary skill in the art and is not to be limited to a special or customized meaning. The term specifically may refer, without limitation, to the at least one item of batch information being related to the at least one known item of position probability information in a manner that for a specific known item of position probability information a specific item of batch information is available. The term “batch” as used herein is a broad term and is to be given its ordinary and customary meaning to a person of ordinary skill in the art and is not to be limited to a special or customized meaning. The term specifically may refer, without limitation, to a quantity of products produced in a uniform manufacturing process. The products may comprise a batch identifier, such as a batch number, that may, typically, be arranged on the packaging and/or housing of the product. By considering the batch information of the optical test element, the accuracy and the reliability may even further be optimized.

For weighting the potential item of intensity information on the optical detection reaction with at least one known item of position probability information on the optical detection reaction, at least one item of batch information on the optical test element may be considered. For considering at least one item of batch information on the optical test element, the at least one known item of position probability information on the optical detection reaction may be selected depending on the at least one item of batch information on the optical test element.

The at least one known item of position probability information, considered for weighting the potential item of intensity information on the optical detection reaction, may be received from at least one of:
a storage device of the mobile device;
a storage device of a providing server, specifically a cloud server, particularly received by using a connection interface of the mobile device.

The term “server” as used herein is a broad term and is to be given its ordinary and customary meaning to a person of ordinary skill in the art and is not to be limited to a special or customized meaning. The term specifically may refer, without limitation, to a device which is capable of performing one or more operations with the data to be processed. Specifically, the server may comprise at least one processor, which may be programmed, by appropriate software, for performing one or more operations with the data to be processed. The term “cloud server” may indicate that the server is a cloud-based server established by at least one computer cloud or a plurality of computers, specifically a server, such as a separate device that receives data from and/or transfers data to the mobile device, particularly via a computer network, such as the internet, more particularly by using at least one respective connection interface. The cloud server may be accessed by the mobile device from a plurality of different mobile device locations. As an example, the connection interface may be configured for providing access of the cloud server and/or of the mobile device to the computer network, specifically in a manner that data may be received and/or transmitted by the respective device via the computer network. The connection interface may be or may comprise at least one port comprising one or more of a network or internet port. In this context, the term “providing” may relate to the fact that an organized collection of data is made available, e.g., by itself, e.g., by providing access to the data, and/or to the process of storing the data in an organized form on the at least one storage device.

A plurality of known items of position probability information, particularly assigned to different items of batch information of the optical test elements, may be stored on the mobile device, wherein the method comprises a step of updating the known plurality of items of position probability information.

The term “updating,” or any grammatical variation thereof, as used herein is a broad term and is to be given its ordinary and customary meaning to a person of ordinary skill in the art and is not to be limited to a special or customized meaning. The term specifically may refer, without limitation, to bringing something to the latest, current state, present condition, or current point in time. Updating may comprise or be an actualization of a computer program and/or a database.

For updating the plurality of items of position probability information, at least one known item of position probability information may be received by the mobile device, particularly from a providing server, specifically a cloud server, more particularly wherein the received at least one known item of position probability information may be stored on the storage device of the mobile device. For updating the plurality of known items of position probability information, at least one known item of position probability information may be deleted from the storage device of the mobile device, particularly wherein the deleted at least one known item of position probability information may be assigned to an item of batch information of optical test elements for which the expiration date has passed.

In a step d., detecting the analyte by considering the weighted potential item of intensity information on the optical detection reaction for differentiating a valid item of intensity information on the optical detection reaction from an invalid item of intensity information, with false positive object.

The term “detecting” as used herein is a broad term and is to be given its ordinary and customary meaning to a person of ordinary skill in the art and is not to be limited to a special or customized meaning. The term specifically may refer, without limitation, to a quantitative and/or qualitative determination of the at least one property of the sample of the bodily fluid, such as at least one of a physical, a chemical and a biological property. The determination of the at least one property may specifically comprise quantitatively or qualitatively detecting at least one analyte in the sample of the bodily fluid. Particularly for generating a result, detecting the analyte may comprise considering of, particularly may be based on, the weighted potential item of intensity information on the optical detection reaction associated with at least one of: the control area; the reagent test region. The potential item of intensity information on the optical detection reaction may be associated with the control area, in case the one or more optical detection features are related to and/or are the result of an optical detection reaction that indicates the correct application of the sample of the bodily fluid on the optical test element, particularly as determined in step d). The potential item of intensity information on the optical detection reaction may be associated with at least one reagent test region, in case the one or more potential item of intensity information on the optical detection reaction are related to and/or are the result of an optical detection reaction that indicates the detection of the analyte, particularly as determined in step d).

The result of the method, as an example, may be at least one item of information on the at least one property of the sample, such as at least one item of information on the presence or absence of an analyte of interest. The result may be indicated to a user of the mobile device, such as by displaying the result on a display or screen comprised by the mobile device. Alternatively or additionally, the result of the method may comprise at least one item of information indicating a concentration of the at least one analyte of interest.

The “analyte” may be or may comprise at least one arbitrary, dedicated and/or predetermined chemical or biological substance or species, such as at least one molecule or at least one chemical and/or biological compound. For example, the analyte may be or may comprise at least one specific virus and/or any parts thereof. The result of the analytical method may be or may comprise at least one item of information indicating the presence or absence of the virus or parts thereof in the sample of the bodily fluid. For example, the analyte may be a chemical compound which takes part in metabolism, such as one or more of glucose, lactate, cholesterol or triglycerides. In this example, the analytical method may be a blood glucose measurement and, thus, the result of the analytical method may, for example, be a blood glucose concentration. Additionally or alternatively, other types of analytes or parameters may be determined, such as a pH value or the like. As will be understood by the skilled person, a “presence” of an analyte, for example, may be a presence of said analyte in an amount above a detection limit of the test used.

The term “differentiating” as used herein is a broad term and is to be given its ordinary and customary meaning to a person of ordinary skill in the art and is not to be limited to a special or customized meaning. The term specifically may refer, without limitation, to the process of distinguishing a plurality of objects based on at least one characteristic of the objects. The distinguished object may be classified based on the considered characteristic.

Differentiating a valid item of intensity information on the optical detection reaction from an invalid item of intensity information, particularly associated with false positive object, may comprise comparing at least one of:
the potential item of intensity information;
the weighted potential item of intensity information;
   to at least one intensity detection threshold, particularly by considering the associated item of position information. The potential item of intensity information may be considered a valid item of intensity information on the optical detection reaction when at least one of:
the potential item of intensity information;
the weighted potential item of intensity information;
   exceeds the intensity detection threshold.

For differentiating a valid item of intensity information on the optical detection reaction from an invalid item of intensity information, particularly associated with false positive object, at least two items of weighted potential items of intensity information on the optical detection reaction may be compared to each other, particularly by considering the associated items of position information. A potential item of intensity information of the at least two items of weighted potential item of intensity information on the optical detection reaction may be considered a valid item of intensity information on the optical detection reaction if said potential item of intensity information exceeds at least one, specifically each, further potential item of intensity information of the at least two items of weighted potential item of intensity information on the optical detection reaction.

As already explained, the image recognition algorithm determines a potential detection of at least one optical detection reaction in the image. Thereby, a potential item of intensity information on the optical detection reaction may be determined that may be associated with an actual optical detection reaction, particularly whereby the term “associated with an actual optical detection reaction” may refer to the fact that the potential item of intensity information on the optical detection reaction is a result of the image recognition algorithm determining information on an optical detection reaction that has actually occurred. Further, a potential item of intensity information on the optical detection reaction may be determined that may not be associated with an actual optical detection reaction and/or that may be associated with a false positive, whereby the term “associated with a false positive” may refer to the fact that the potential item of intensity information on the optical detection reaction is a result of the image recognition algorithm determining information on a false positive, such as a falsely identified feature in the image and/or an artifact in the image.

The term “false positive” as used herein is a broad term and is to be given its ordinary and customary meaning to a person of ordinary skill in the art and is not to be limited to a special or customized meaning. The term specifically may refer, without limitation, to a feature in the image that is identified incorrectly as being associated with the optical detection reaction. Such false positives may be at least one of: an artifact in the image, a falsely identified feature in the image. The “artifact” may be at least one distortion and/or anomaly that may occur during the image acquisition and/or processing. Typical artifacts may be caused by noise generated the camera, camera specific issues, such as chromatic aberration, and/or handling the image data, such as a compression. The “falsely identified feature in the image” may be at least one arbitrary ‘real-life’ object and/or structure that is visible in the image, which is similar the visible optical detection reaction in terms of at least one criteria applied by the image recognition algorithm for recognizing the visible optical detection reaction. The falsely identified feature may be a shadow.

In a further aspect, this disclosure relates to a mobile device having at least one camera, and, optionally, at least one processor, the mobile device being configured, specifically by software configuration, for performing the computer-implemented method of determining at least one analyte in a sample of a bodily fluid. For details, options and definitions, reference may be made to any aspect, embodiment or claim discussed elsewhere herein.

The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming.

In a further aspect, this disclosure relates to a computer program comprising instructions which, when the program is executed by the mobile device, cause the mobile device to perform the computer-implemented method of determining at least one analyte in a sample of a bodily fluid. For details, options and definitions, reference may be made to any aspect, embodiment or claim discussed elsewhere herein. Specifically, the computer program may be stored on a computer-readable data carrier and/or on a computer-readable storage medium.

In a further aspect, this disclosure relates to a computer-readable storage medium, specifically a non-transient computer-readable storage medium, comprising instructions which, when the instructions are executed by the mobile device, cause the mobile device to perform the computer-implemented method of determining at least one analyte in a sample of a bodily fluid. For details, options and definitions, reference may be made to any aspect, embodiment or claim discussed elsewhere herein. As used herein, the terms “computer-readable data carrier” and “computer-readable storage medium” specifically may refer to non-transitory data storage means, such as a hardware storage medium having stored thereon computer-executable instructions. The computer-readable storage medium specifically may be or may comprise a storage medium such as a random-access memory (RAM) and/or a read-only memory (ROM). For details, options and definitions, reference may be made to any further aspect discussed elsewhere herein.

In a further aspect, this disclosure relates to an analytical system comprising:
at least one mobile device, wherein the mobile device is having at least one camera, and
at least one optical test element having at least one test field configured for performing at least one optically detectable detection reaction in the presence of at least one analyte, particularly in a sample of bodily fluid applied to the optical test element.

For details, options and definitions, reference may be made to any aspect, embodiment or claim discussed elsewhere herein.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. The system may comprise multiple components, for example, at least two components or even more. The at least two components may be handled independently or may be coupled or connectable. The components of the system may interact with each other in order to fulfill at least one common function. The term “analytical system,” may refer to a system for performing at least one analytical function, specifically for performing at least one analytical measurement, such as by performing the above described method. The analytical system may be further comprising at least one optical test element having at least one test field configured for performing at least one optically detectable detection reaction in the presence of at least one analyte, particularly in a sample of bodily fluid applied to the optical test element.

As an example, a computer-implemented method of detecting an analyte (concentration) in a sample of bodily fluid by using a mobile device having a camera, may comprise:
a) identifying preliminary (positive) detection results by applying a (threshold-based) line detection algorithm to an image, captured by the camera, of a test element which comprises, in a test area, one or more test (and/or control) lines, said lines resulting from a chemical reaction of an analyte contained in the sample, the sample having been applied to the test element; and
b) weighting the preliminary positive detection results of step a) with known probabilities of actual manufacturing tolerances, said tolerances relating to the (optionally lot-specific) actual position(s) in the test area where the one or more test (and/or control) lines are expected to be observable, thereby obtaining final (positive) detection results representing only valid test (and/or control) lines.

Thus, specifically, one, more than one or even all of method steps a. to d. as indicated above may be performed by using a computer or a computer network, preferably by using a computer program.

Further disclosed and proposed herein is a computer program product having program code means, in order to perform the method according to this disclosure in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of this disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

The methods and devices according to this disclosure may provide a large number of advantages compared with known methods and devices.

Typically, a photo app may used on a mobile device for detecting an analyte via the mobile device, in particular in home-testing scenarios. Therefore, at least one image of an optical test element, such as a lateral flow test assay, specifically for a rapid antigen test, may be captured by a camera comprised by the mobile device. By considering at least one visible optical detection reaction the presence and/or concentration of the analyte may be determined. Particularly in the sense, the optical detection reaction may cause a detectable visible change resulting in a visible feature that is introduced by the optical detection reaction within a test field comprised by the optical test element. Through the course of the optical detection reaction the visible features may appear. Such features may specifically be graphical elements, such as at least one bar, test-lines, and/or a color formation.

Typically, an image recognition based feature detection algorithm is applied. The feature detection algorithm may receive an image from a camera of the mobile device and may search for the feature, exemplary at least one line. If the feature is found, the feature detection algorithm may return an intensity and a position for the found feature. In some cases, however, false positives, such as artifacts, may be present in the image that may be categorized as a feature. Exemplarily, an inhomogeneity in the illumination of the test field and/or a shadow that is cast on the test field, may be falsely identified as a feature, such as a test-line.

Particularly in order to differentiate a feature from false positive in the image an enhanced feature detection algorithm is proposed. In this enhanced feature detection algorithm, the position of a potential feature determined by the image recognition algorithm may be compared with a predetermined nominal position of the feature that is provided by the manufacturer, exemplarily taking into account actual manufacturing tolerances that may, exemplarily, be given specific for manufacturing batches. Particularly for comparing, the determined intensity of the potential feature may be weight with a known position probability. In that manner the features detected close to the predetermined nominal position may be categorized as valid features associated with the optical detection reaction. Thereby, more accurate and reliable test results may be achieved, particularly for Covid-19 tests.

Particularly for better a feature detection performance, it is thus proposed to take manufacturing tolerances into account, particularly batch specific manufacturing tolerances, whereby the feature detection algorithm combines the intensity of a potential optical detection related feature with a position probability that considers manufacturing tolerances.

This disclosure may thus provide an enhanced reliability of analyte testing based on a visible optical detection reaction. The presented approach may be more accurate than relying on theoretical manufacturing tolerances only, particularly in case a calibration process is performed. It may further be more accurate than a visual inspection by user. Additionally, less test results may be discarded as invalid. This disclosure is, thereby, more reliable than an approach that relies on a consideration of a static region of interest related to the optical test element in the image having a constant feature detection algorithm threshold.

A concrete use of the manufacturing tolerances may be in the implementation of a mobile device based readout module of a lateral flow Covid-19 tests. The manufacturer of the Covid-19 optical test element may provide a predetermined position on the test field comprising a tolerance on the a control line feature as well as a test line feature.

The image recognition algorithm receives an image of the camera of the mobile device and searches for lines. If a line is found, the image recognition algorithm returns the line's intensity and associated position. In some cases artifacts present in the images may be categorized as feature lines. Furthermore, a possible inhomogeneity in the illumination of the test field or shadows that are cast on the test field may be falsely identified as a test line feature. One way to discriminate test lines from artifacts may be comparing the position of the line estimated by the algorithm to the one provided by the manufacturer.

Summarizing and without excluding further possible embodiments, the following embodiments may be envisaged:

Embodiment 1: A computer-implemented method of detecting at least one analyte in a sample of a bodily fluid by using a mobile device having at least one camera, the method comprising:
a. obtaining image data of at least one image captured by using the camera, wherein the image comprises at least a part of a test field of an optical test element having the sample of the bodily fluid applied thereto, wherein the test field is configured for performing at least one optical detection reaction in the presence of the analyte in the sample of the bodily fluid, wherein the optical detection reaction is visible at at least one predetermined position on the test field;
b. performing at least one image recognition algorithm on the image, wherein the image recognition algorithm is configured for determining at least one potential item of intensity information on the optical detection reaction in the image and at least one associated item of position information on the optical detection reaction in the image;
c. weighting the potential item of intensity information on the optical detection reaction with at least one known item of position probability information on the optical detection reaction, wherein the known item of position probability information on the optical detection reaction is determined by considering the associated item of position information on the optical detection reaction, wherein the at least one known item of position probability information considers at least one tolerance in a manufacturing process of the optical test element having an influence on the at least one predetermined position of the optical detection reaction on the test field;
d. detecting the analyte by considering the weighted potential item of intensity information on the optical detection reaction for differentiating a valid item of intensity information on the optical detection reaction from an invalid item of intensity information, particularly associated with false positive.

Embodiment 2: The computer-implemented method according to the preceding Embodiment, wherein the method comprises a step of capturing the at least one image by using the camera.

Embodiment 3: The computer-implemented method according to any one of the preceding Embodiments, wherein the optical detection reaction comprises a reaction during which one or more optical detection features on or within the optical test element becomes visible.

Embodiment 4: The computer-implemented method according to the preceding Embodiment, wherein the one or more optical detection features are associated with at least one of:
at least one control area configured for indicating a correct application of the sample of the bodily fluid on the optical test element;
at least one reagent test region for detecting the analyte,
   particularly wherein the control area is at a different predetermined position than the reagent test region.

Embodiment 5: The computer-implemented method according to the preceding Embodiment, wherein detecting the analyte comprises considering of, particularly is based on, the weighted potential item of intensity information on the optical detection reaction associated with at least one of:
the control area;
the reagent test region.

Embodiment 6: The computer-implemented method according to the three preceding Embodiments, wherein the one or more optical detection features are one or more linear markings.

Embodiment 7: The computer-implemented method according to any one of the preceding Embodiments, wherein the optical detection reaction comprises a color formation reaction.

Embodiment 8: The computer-implemented method according to any one of the preceding Embodiments, wherein detecting the analyte comprises determining at least one concentration of the analyte.

Embodiment 9: The computer-implemented method according to any one of the preceding Embodiments, wherein the image recognition algorithm determines the at least one associated item of position information by considering at least one of:
a position of a maximum of an intensity distribution, particularly comprised in the potential item of intensity information;
a position of a center of an intensity distribution, particularly comprised in the potential item of intensity information;
a position of a centroid of an intensity distribution, particularly comprised in the potential item of intensity information,
   particularly for finding a position of the associated item of position information in reference to the image.

Embodiment 10: The computer-implemented method according to any one of the preceding Embodiments, wherein the known item of position probability information refers to a probability of a potential item of intensity information on the optical detection reaction to be a valid item of intensity information on the optical detection reaction depending on the associated item of position information on the optical detection.

Embodiment 11: The computer-implemented method according to any one of the preceding Embodiments, wherein the at least one known item of position probability information is comprised by a distribution of known position probabilities.

Embodiment 12: The computer-implemented method according to any one of the preceding Embodiments, wherein at least one item of position information of a position indicating feature is determined in the image, particularly by performing the image recognition algorithm, wherein the associated item of position information on the optical detection reaction is determined by considering the item of position information on the position indicating feature, particularly for finding a position of the associated item of position information in reference to the optical test element.

Embodiment 13: The computer-implemented method according to of the preceding Embodiment, wherein, for considering the item of position information on the position indicating feature when determining the associated item of position information on the optical detection reaction, a relative position, particularly a distance, between the item of position information of a position indicating feature and the associated item of position information on the optical detection reaction is considered.

Embodiment 14: The computer-implemented method according to any one of the two preceding Embodiments, wherein the item of position information on the position indicating feature is determined by considering at least one of:
at least one position of at least one marking on the optical test element;
at least one position of at least one geometric feature of the optical test element, particularly at least one feature of a window of the optical test element showing the test field;
at least one position of at least one optical detection feature generated by the optical detection reaction, specifically a position of a control area or a position of a reagent test region;
   comprised by the image.

Embodiment 15: The computer-implemented method according to any one of the preceding Embodiments, wherein differentiating a valid item of intensity information on the optical detection reaction from an invalid item of intensity information, particularly associated with false positive, comprises comparing at least one of:
the potential item of intensity information;
the weighted potential item of intensity information;
to at least one intensity detection threshold.

Embodiment 16: The computer-implemented method according to the preceding Embodiment, wherein the potential item of intensity information is considered a valid item of intensity information on the optical detection reaction when at least one of:
the potential item of intensity information;
the weighted potential item of intensity information;
exceeds the intensity detection threshold.

Embodiment 17: The computer-implemented method according to any one of the preceding Embodiments, wherein, for differentiating a valid item of intensity information on the optical detection reaction from an invalid item of intensity information, particularly associated with false positive, at least two items of weighted potential items of intensity information on the optical detection reaction are compared to each other.

Embodiment 18: The computer-implemented method according to the preceding Embodiment, wherein a potential item of intensity information of the at least two items of weighted potential item of intensity information on the optical detection reaction is considered a valid item of intensity information on the optical detection reaction if said potential item of intensity information exceeds at least one, specifically each, further potential item of intensity information of the at least two items of weighted potential item of intensity information on the optical detection reaction.

Embodiment 19: The computer-implemented method according to any one of the preceding Embodiments, wherein, for weighting the potential item of intensity information on the optical detection reaction with at least one known position probability information, the potential item of intensity information on the optical detection reaction is at least one of:
convoluted;
multiplied;
   with the known item of position probability information.

Embodiment 20: The computer-implemented method according to any one of the preceding Embodiments, wherein the at least one known item of position probability information considering the at least one tolerance in the manufacturing process of the optical test element having an influence on the at least one predetermined position on the test field is determined in a calibration process, particularly a factory calibration process.

Embodiment 21: The computer-implemented method according to the preceding Embodiment, wherein in the calibration process at least one item of batch information on the optical test element assigned to the at least one known item of position probability information is determined.

Embodiment 22: The computer-implemented method according to any one of the preceding Embodiments, wherein, for weighting the potential item of intensity information on the optical detection reaction with at least one known item of position probability information on the optical detection reaction, at least one item of batch information on the optical test element is considered.

Embodiment 23: The computer-implemented method according to the preceding Embodiment, wherein, for considering at least one item of batch information on the optical test element, the at least one known item of position probability information on the optical detection reaction is selected depending on the at least one item of batch information on the optical test element.

Embodiment 24: The computer-implemented method according to any one of the preceding Embodiments, wherein the at least one known item of position probability information, considered for weighting the potential item of intensity information on the optical detection reaction, is received from at least one of:
a storage device of the mobile device;
a storage device of a providing server, specifically a cloud server, particularly received by using a connection interface of the mobile device.

Embodiment 25: The computer-implemented method according to any one of the preceding Embodiments, wherein a plurality of known items of position probability information, particularly assigned to different items of batch information of the optical test elements, is stored on the mobile device, wherein the method comprises a step of updating the known plurality of items of position probability information.

Embodiment 26: The computer-implemented method according to the preceding Embodiments, wherein, for updating the plurality of items of position probability information, at least one known item of position probability information is received by the mobile device, particularly from a providing server, specifically a cloud server, more particularly wherein the received at least one known item of position probability information is stored on the storage device of the mobile device.

Embodiment 27: The computer-implemented method according to any one of the two preceding Embodiments, wherein, for updating the plurality of known items of position probability information, at least one known item of position probability information is deleted from the storage device of the mobile device, particularly wherein the deleted at least one known item of position probability information is assigned to an item of batch information of optical test elements for which the expiration date has passed.

Embodiment 28: A mobile device having at least one camera, and, optionally, at least one processor, the mobile device being configured, specifically by software configuration, for performing the computer-implemented method of determining at least one analyte in a sample of a bodily fluid according to any one of the preceding Embodiments referring to a computer-implemented method.

Embodiment 29: A computer program comprising instructions which, when the program is executed by the mobile device according to the preceding Embodiment, cause the mobile device to perform the computer-implemented method of determining at least one analyte in a sample of a bodily fluid according to any one of the preceding Embodiments referring to a computer-implemented method.

Embodiment 30: A computer-readable storage medium, specifically a non-transient computer-readable storage medium, comprising instructions which, when the instructions are executed by the mobile device according to the Embodiment referring to a mobile device, cause the mobile device to perform the computer-implemented method of determining at least one analyte in a sample of a bodily fluid according to any one of the preceding Embodiments referring to a computer-implemented method.

Embodiment 31: An analytical system comprising:
at least one mobile device according to the Embodiment referring to at least one mobile device, wherein the mobile device is having at least one camera, and
at least one optical test element having at least one test field configured for performing at least one optically detectable detection reaction in the presence of at least one analyte, particularly in a sample of bodily fluid applied to the optical test element.

### BRIEF DESCRIPTION OF THE DRAWINGS

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of this disclosure is not restricted by the preferred embodiments. The embodiments are schematically depicted in the Figures. Therein, identical reference numbers in these Figures refer to identical or functionally comparable elements.

The above-mentioned aspects of exemplary embodiments will become more apparent and will be better understood by reference to the following description of the embodiments taken in conjunction with the accompanying drawings, wherein:

FIG. 1 shows an exemplary optical test element;

FIG. 2 shows a detailed view of a test field region of the exemplary optical test element with a sample of a bodily fluid applied thereon;

FIGS. 3a and 3b show exemplary position probability distributions associated with a control area and a reagent test region of the exemplary optical test element;

FIG. 4 shows an exemplary mobile device used for detecting the at least one analyte in the sample of the bodily fluid;

FIG. 5 shows a computer-implemented method of detecting at least one analyte in a sample of a bodily fluid; and

FIGS. 6a and 6b show the impact of weighting the item of potential intensity information for detecting the analyte.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

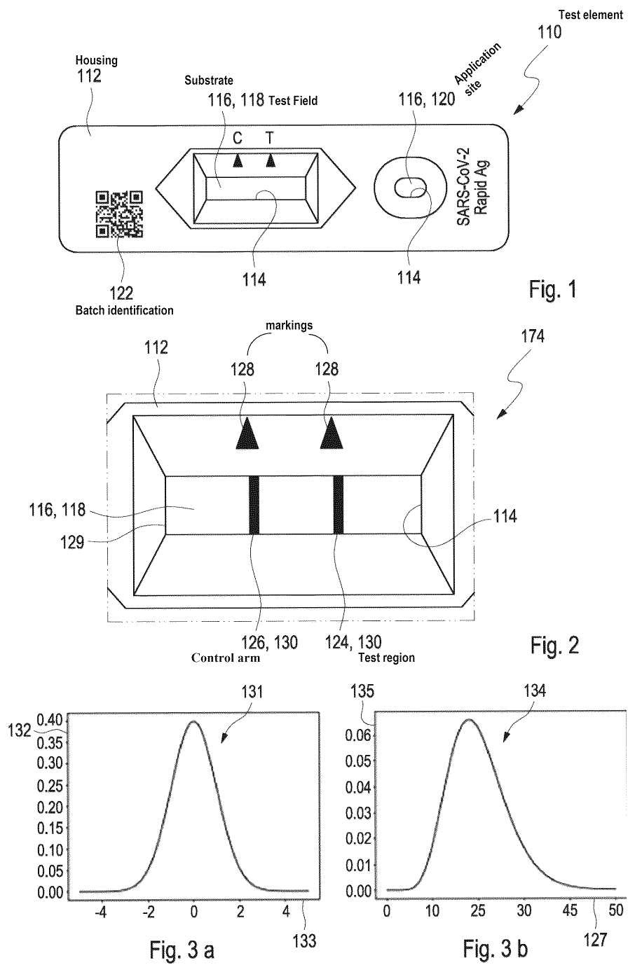

FIG. 1 shows an exemplary optical test element 110 for performing lateral flow Covid-19 tests, which may be a typical use case of this disclosure, particularly referring to detecting at least one analyte in a sample of a bodily fluid by using a mobile device 136 having at least one camera 138. The optical test element 110 may comprise a housing 112. The optical test element 110, particularly the housing 112, may further comprise at least one window 114 through which access to a substrate 116 may be possible. The substrate 116 may comprise a test field 118 and/or an application site 120. The optical test element 110 further comprises a batch identification feature 122.

FIG. 2 shows an image 174 of the test field 118 region of the optical test element 110 with a sample of a bodily fluid applied thereon so that an optical detection reaction took place as a result of which a reagent test region 124 and a control area 126 became visible. The optical detection reaction may comprise a color formation reaction. The optical detection reaction may comprise a reaction during which one or more optical detection features 130 on or within the optical test element 110 becomes visible. The one or more optical detection features 130 may be one or more linear markings 128. The one or more optical detection features 130 may be associated with at least one of: at least one control area 126 configured for indicating a correct application of the sample of the bodily fluid on the optical test element 110; at least one reagent test region 124 for detecting the analyte, particularly wherein the control area 126 is at a different predetermined position than the reagent test region 124. The position of the feature 130 associated with the reagent test region 124 and the feature 130 associated with the control area 126 is indicated by markings 128 on the housing 112. The optical test element 110 may further comprise at least one geometric feature 129. The at least one geometric feature 129 may be a feature of the window 114, such as an edge of the window 114, of the optical test element 110 showing the test field 118. Particularly due to tolerances in the manufacturing process, there may be an offset between the position of the optical detection reaction occurring in the reagent test region 124 and the control area 126. Alternatively or in addition, there may be a, particularly common, offset of the feature 130 associated with the at least one control area 126 and the feature 130 associated at least one reagent test region 124 in respect to at least one of: the housing 112; the window 114; the at least one geometric feature 129, particularly due to a positional manufacturing tolerance of the substrate 116 in the housing 112.

FIG. 3a shows an exemplary position probability distribution 131 determined in a factory calibration process for the feature 130 associated with the control area 126. On a horizontal axis 133, a position of the feature 130 associated with, particularly a feature 130 in, the control area 126 is indicated. On a vertical axis 132, a probability of a valid feature 130 to be at the position is indicated. FIG. 3b shows an exemplary position probability distribution 134 determined in a factory calibration process for the feature 130 associated with the reagent test region 124. On a horizontal axis 127, a position of the feature 130 associated with, particularly a feature 130 in, the reagent test region 124 is indicated. On a vertical axis 135, a probability of a valid feature 130 to be at the position is indicated. At least one of the position probability distribution 131 and position probability distribution 134 may be provided by the manufacturer.

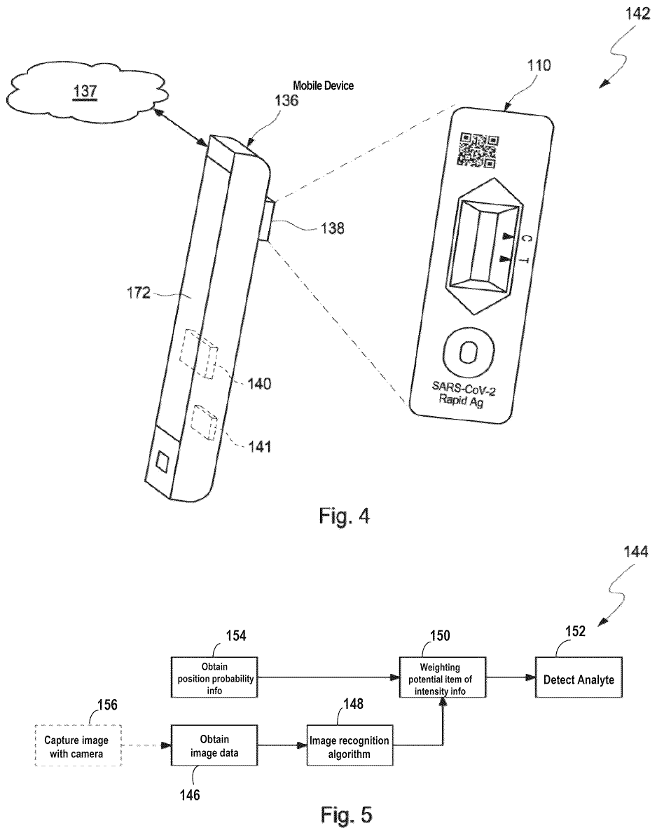

As may be derived from FIG. 4, in an exemplary mobile device 136, particularly used for detecting the at least one analyte in the sample of the bodily fluid, the mobile device 136 has at least one camera 138. The mobile device 136 may further have at least one processor 140. The mobile device 136 may comprise a storage device 141. The mobile device 136 is configured, specifically by software configuration, for performing at least one computer-implemented method of detecting at least one analyte in a sample of a bodily fluid as described in the following. Particularly for configuring the mobile device 136 by software configuration, a computer program, comprising instructions which, when the program is executed by the mobile device 136, cause the mobile device 136 to perform the computer-implemented method of determining at least one analyte in a sample of a bodily fluid. Particularly the computer program may be stored on a computer-readable storage medium. The computer-readable storage medium, specifically a non-transient computer-readable storage medium, may comprise instructions which, when the instructions are executed by the mobile device 136, cause the mobile device 136 to perform the computer-implemented method of determining at least one analyte in a sample of a bodily fluid.

As may further be derived from FIG. 4, the mobile device 136 may transfer data to and/or receive data from a server 137, particularly by using a connection interface of the mobile device 136.

As may further be derived from FIG. 4,
the at least one mobile device 136 having at least one camera 138, and
the at least one optical test element 110 having at least one test field 118 configured for performing at least one optically detectable detection reaction in the presence of at least one analyte, particularly in a sample of bodily fluid applied to the optical test element 110,
   are comprised by an analytical system 142.

In FIG. 5, an exemplary computer-implemented method 144 of detecting at least one analyte in a sample of a bodily fluid by using a mobile device 136 having at least one camera 138 is illustrated. The method comprises:
a. in a step 146, obtaining image data of at least one image 174 captured by using the camera 138, wherein the image 174 comprises at least a part of a test field 118 of an optical test element 110 having the sample of the bodily fluid applied thereto, wherein the test field 118 is configured for performing at least one optical detection reaction in the presence of the analyte in the sample of the bodily fluid, wherein the optical detection reaction is visible at at least one predetermined position on the test field 118;
b. in a step 148, performing at least one image recognition algorithm on the image 174, particularly a line detection algorithm, wherein the image recognition algorithm is configured for determining a potential detection of at least one optical detection feature of at least one optical detection reaction in the image 174, wherein the image recognition algorithm is configured for determining at least one potential item of intensity information on the optical detection feature of the optical detection reaction in the image 174 and at least one associated item of position information on the optical detection feature of the optical detection reaction in the image 174;
c. in a step 150, weighting the potential item of intensity information on the optical detection feature of the optical detection reaction with at least one known item of position probability information on the optical detection reaction, particularly obtained in a step 154, wherein the known item of position probability information is a probability of a potential item of intensity information on the optical detection reaction to be a valid item of intensity information on the optical detection reaction depending on the associated item of position information on the optical detection, wherein the known item of position probability information on the optical detection reaction is determined by considering the associated item of position information on the optical detection reaction, wherein the at least one known item of position probability information considers at least one tolerance in a manufacturing process of the optical test element 110 having an influence on the at least one predetermined position of the optical detection reaction on the test field 118;
d. in a step 152, detecting the analyte by considering the weighted potential item of intensity information on the optical detection reaction for differentiating a valid item of intensity information on the optical detection reaction from an invalid item of intensity information, particularly associated with false positive.

The method may further comprise a step 156 of capturing the at least one image 174 by using the camera 138.

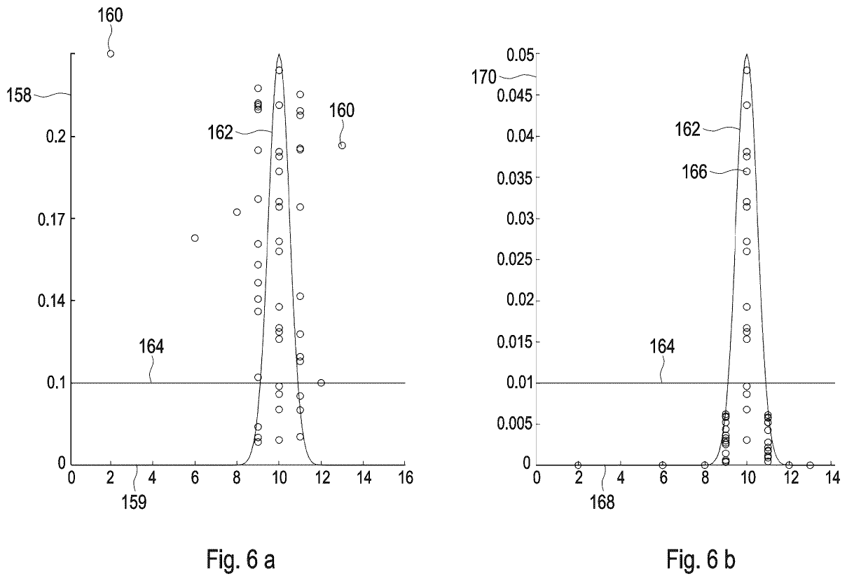

For deriving FIG. 6a, a group of images 174 was assessed by using the image recognition algorithm. The image recognition algorithm may determine at least one potential event 160 (depicted as circles in FIG. 6a), particularly associated with an item of potential intensity information on the optical detection reaction and an associated item of position information. The image recognition algorithm, particularly in the step 148, may determine the at least one associated item of position information by considering at least one of:
a position of a maximum of an intensity distribution, particularly comprised in the potential item of intensity information;
a position of a center of an intensity distribution, particularly comprised in the potential item of intensity information;
a position of a centroid of the intensity distribution, particularly comprised in the potential item of intensity information,
   particularly for finding a position of the associated item of position information in reference to the image 174.

On the vertical axis 158 in FIG. 6a, the intensity information of a potential item on the optical detection reaction, particularly a feature, specifically a line, is illustrated in arbitrary units. On the horizontal axis 159 in FIG. 6a, the associated position information is illustrated in arbitrary units. In FIG. 6a, further a distribution 162 of the known position probabilities and a detection threshold 164 are depicted. It may be noticed that some of the determined items of potential intensity information on the optical detection reaction and the associated item of position information line positions, lie outside the distribution 162 of the known position probabilities, particularly thereby indicating a false positive.

The origin of the horizontal axis 159 in FIG. 6a may associated with at least one item of position information on a position indicating feature 128, 129, 130. The at least one item of position information of the position indicating feature 128, 129, 130 may be determined in the image 174, particularly by performing the image recognition algorithm, wherein the associated item of position information on the optical detection reaction may be determined by considering the item of position information on the position indicating feature 128, 129, 130, particularly for finding a position of the associated item of position information in reference to the optical test element 110. For considering the item of position information on the position indicating feature 128, 129, 130 when determining the associated item of position information on the optical detection reaction, a relative position, particularly a distance, between the item of position information of a position indicating feature 128, 129, 130 and the associated item of position information on the optical detection reaction may be considered.

The item of position information on the position indicating feature 128, 129, 130 may be determined by considering at least one of:
at least one position of at least one marking 128 on the optical test element 110, particularly whereas an item associated item of position information may be given for a feature 130 associated with control area 126 and/or the reagent test region 124;
at least one position of at least one geometric feature 129 of the optical test element 110, particularly at least one feature of a window 114 of the optical test element 110 showing the test field 118, particularly whereas an item associated item of position information may be given for a feature 130 associated with control area 126 and/or the reagent test region 124;
at least one position of at least one optical detection feature 130 generated by the optical detection reaction, specifically a position of a control area 126 or a position of a reagent test region 124, particularly whereas the associated item of position information may be given relative to the at least one position of at least one optical detection feature 130 generated by the optical detection reaction;
   comprised by the image 174.

FIG. 6b illustrates the same measurement data but with at least one weighted event 166 (depicted as circles in FIG. 6b), particularly comprising a weighted potential item of intensity information on the optical detection reactions and the associated item of position information. On the vertical axis 170, the intensity information of a potential item on the optical detection reaction, particularly a feature 130, specifically a line, is illustrated in arbitrary units. On the horizontal axis 168, the associated position information is illustrated in arbitrary units.

For weighting the potential item of intensity information on the optical detection reaction with at least one known position probability information, the potential item of intensity information on the optical detection reaction may be at least one of: convoluted; multiplied; with the known item of position probability information. For weighting the potential item of intensity information on the optical detection reaction with at least one known item of position probability information on the optical detection reaction, at least one item of batch information on the optical test element 110 may be considered. For considering at least one item of batch information on the optical test element 110, the at least one known item of position probability information on the optical detection reaction may be selected depending on the at least one item of batch information on the optical test element 110.

The known item of position probability information may refer to a probability of a potential item of intensity information on the optical detection reaction to be a valid item of intensity information on the optical detection reaction depending on the associated item of position information on the optical detection. The at least one known item of position probability information may be comprised by a distribution of known position probabilities. The at least one known item of position probability information considering the at least one tolerance in the manufacturing process of the optical test element 110 having an influence on the at least one predetermined position on the test field 118 may be determined in a calibration process, particularly a factory calibration process. In the calibration process at least one item of batch information on the optical test element 110 assigned to the at least one known item of position probability information may be determined.

The at least one known item of position probability information, considered for weighting the potential item of intensity information on the optical detection reaction, may be received from at least one of:
a storage device of the mobile device 136;
a storage device of a providing server 137, specifically a cloud server 137, particularly received by using a connection interface of the mobile device 136.

A plurality of known items of position probability information, particularly assigned to different items of batch information of the optical test elements 110, may be stored on the mobile device 136, wherein the method comprises a step of updating the known plurality of items of position probability information.

For updating the plurality of items of position probability information, at least one known item of position probability information may be received by the mobile device 136, particularly from a providing server 137, specifically a cloud server 137, more particularly wherein the received at least one known item of position probability information may be stored on the storage device of the mobile device 136.

For updating the plurality of known items of position probability information, at least one known item of position probability information may be deleted from the storage device of the mobile device 136, particularly wherein the deleted at least one known item of position probability information may be assigned to an item of batch information of optical test elements 110 for which the expiration date has passed.

Only the weighted potential items of intensity information on the optical detection reactions detected close to a predetermined expected value may be categorized as valid items of intensity information on the optical detection reaction, particularly thereby yielding a positive for Covid-19. Particularly in the step 152, differentiating a valid item of intensity information on the optical detection reaction from an invalid item of intensity information, particularly associated with a false positive, may comprise comparing at least one of:
the potential item of intensity information;
the weighted potential item of intensity information;
   to at least one intensity detection threshold, particularly by considering the associated item of position information. The potential item of intensity information may be considered a valid item of intensity information on the optical detection reaction when at least one of:
the potential item of intensity information;
the weighted potential item of intensity information;
   exceeds the intensity detection threshold.

Particularly in the step 152, for differentiating a valid item of intensity information on the optical detection reaction from an invalid item of intensity information, particularly associated with false positive, at least two items of weighted potential items of intensity information on the optical detection reaction may be compared to each other, particularly by considering the associated items of position information. A potential item of intensity information of the at least two items of weighted potential item of intensity information on the optical detection reaction may be considered a valid item of intensity information on the optical detection reaction if said potential item of intensity information exceeds at least one, specifically each, further potential item of intensity information of the at least two items of weighted potential item of intensity information on the optical detection reaction.

Detecting the analyte, particularly in the step 152, may comprise considering of, particularly may be based on, the weighted potential item of intensity information on the optical detection reaction associated with at least one of: the control area 126; the reagent test region 124. Detecting the analyte, particularly in the step 152, may comprise determining at least one concentration of the analyte. When determining the concentration of the analyte, the item of intensity information may be considered. The result of the detection may be displayed on a display 172 of the mobile device 136.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

LIST OF REFERENCE NUMBERS

| | |
|---|---|
| 110 | optical test element |
| 112 | housing |
| 114 | window |
| 116 | substrate |
| 118 | test field |
| 120 | application site |
| 122 | batch identification feature |
| 124 | reagent test region |
| 126 | control area |
| 127 | horizontal axis |
| 128 | marking |
| 129 | geometric feature |
| 130 | feature |
| 131 | position probability distribution associated with the control area |
| 132 | vertical axis |
| 133 | horizontal axis |
| 134 | position probability distribution associated with the reagent test |
| | region |
| 135 | vertical axis |
| 136 | mobile device |
| 137 | server |
| 138 | camera |
| 140 | processor |
| 141 | storage device |
| 142 | analytical system |
| 144 | computer-implemented method |
| 146 | obtaining image data of at least one image captured by using the |
| | camera |
| 148 | performing at least one image recognition algorithm on the image |
| 150 | weighting the potential item of intensity information |
| 152 | detecting the analyte |
| 154 | obtaining at least one known item of position probability |
| | information |
| 156 | capturing the at least one image by using the camera |
| 158 | vertical axis |
| 159 | horizontal axis |
| 160 | potential feature |
| 162 | distribution of the known position probabilities |
| 164 | detection threshold |
| 166 | weighted event |
| 168 | horizontal axis |
| 170 | vertical axis |
| 172 | display |
| 174 | image |

What is claimed is:

1. A method of detecting an analyte in a sample of body fluid using a mobile device having a camera, the method comprising:
a. obtaining image data of an image captured with the camera, the image comprising at least part of a test field of an optical test element having the sample of the body fluid applied thereto, wherein the test field is configured for performing an optical detection reaction in the presence of the analyte in the body fluid and the optical detection reaction is visible at a predetermined position on the test field;
b. performing, on the image, an image recognition algorithm configured for determining a potential detection of an optical detection feature of an optical detection reaction in the image, the image recognition algorithm further configured for determining a potential item of intensity information on the optical detection feature and an item of position information on the optical detection feature;
c. weighting the potential item of intensity information with a known item of position probability information on the optical detection reaction, wherein the known item of position probability information is a probability of a potential item of intensity information on the optical detection reaction being a valid item of intensity information on the optical detection reaction depending on the associated item of position information on the optical detection, wherein the known item of position probability information is determined by considering the associated item of position information, wherein the known item of position probability information considers a tolerance in a manufacturing process of the optical test element having an influence on the predetermined position of the optical detection reaction on the test field;
d. detecting the analyte by considering the weighted potential item of intensity information on the optical detection reaction for differentiating a valid item of intensity information on the optical detection reaction from an invalid item of intensity information;
wherein in a calibration process an item of batch information on the optical test element assigned to the known item of position probability information is determined in a manner that for a specific known item of position probability information a specific item of batch information is available, wherein for weighting the potential item of intensity information with the known item of position probability information, the known item of position probability information is selected depending on the item of batch information.

2. The method according to claim 1, further comprising the step of capturing the image by using the camera.

3. The method according to claim 1, wherein the optical detection reaction comprises a reaction during which one or more optical detection features on or within the optical test element becomes visible, wherein the one or more optical detection features are associated with at least one of:
a control area configured for indicating a correct application of the sample on the optical test element;
a reagent test region for detecting the analyte.

4. The method according to claim 1, wherein a item of position information of a position indicating feature is determined in the image, wherein the associated item of position information on the optical detection reaction is determined by considering the item of position information on the position indicating feature, wherein the item of position information on the position indicating feature is determined by considering at least one of:
a position of a marking on the optical test element;
a position of a geometric feature of the optical test element;
a position of an optical detection feature generated by the optical detection reaction.

5. The method according to claim 1, wherein differentiating a valid item of intensity information on the optical detection reaction from an invalid item of intensity information comprises comparing at least one of (i) the potential item of intensity information and (ii) the weighted potential item of intensity information to a detection threshold.

6. The method according to claim 1, wherein, for differentiating a valid item of intensity information on the optical detection reaction from an invalid item of intensity information, at least two items of weighted potential items of intensity information on the optical detection reaction are compared to each other.

7. The method according to claim 1, wherein the known item of position probability information considering the tolerance in the manufacturing process of the optical test element having an influence on the predetermined position on the test field is determined in the calibration process.

8. The method according to claim 7, wherein in the calibration process at least one item of batch information on the optical test element assigned to the known item of position probability information is determined.

9. The method according to claim 1, wherein a plurality of known items of position probability information is stored on the mobile device, wherein the method comprises updating the known plurality of items of position probability information.

10. The method according to claim 9, wherein, for updating the plurality of items of position probability information, a known item of position probability information is received by the mobile device from a providing server.

11. A mobile device having at least one camera and at least one processor, the mobile device being configured for performing the method of claim 1.

12. A computer program comprising instructions which, when the program is executed by the mobile device according to claim 11, cause the mobile device to perform the computer-implemented method of claim 1.

13. The mobile device according to claim 11, further comprising a camera and an optical test element having a test field configured for performing an optically detectable detection reaction in the presence of an analyte in a sample of body fluid applied to the optical test element.

14. A non-transitory computer readable medium having stored thereon computer-executable instructions for performing the method according to claim 1.
